(19) United States
(12) Patent Application Publication
(10) Pub. No.: US 2016/0157842 A1
(43) Pub. Date: Jun. 9, 2016

(54) SURGICAL INSTRUMENT AND METHOD OF USE THEREOF

(71) Applicants: Peter KURZWEIL, Memphis, TN (US); Smith & Nephew, Inc., Memphis, TN (US)

(72) Inventors: Peter Kurzweil, Memphis, TN (US); Jeffrey Wyman, Naples, FL (US); Stephen Santangelo, Sturbridge, MA (US); Oliver Streit, Tuttlingen (DE)

(21) Appl. No.: 14/900,362

(22) PCT Filed: Jun. 20, 2014
(86) PCT No.: PCT/US2014/043434

§ 371 Date: Dec. 21, 2015

Related U.S. Application Data
(60) Provisional application No. 61/838,198, filed on Jun. 21, 2013.

(51) Int. Cl. A61B 17/00; A61B 1/317

(52) U.S. Cl. CPC A61B 17/00234; A61B 1/317; A61B 2090/3618

(57) ABSTRACT

A surgical instrument for use during arthroscopic surgery comprising an elongated member, a handle, a tool disposed at the opposite end from the handle of the elongated member, and a reflecting member disposed along the elongated member and configured to reflect a line of sight of an arthroscope during arthroscopic surgery. Methods of using the surgical instrument, and a sterilizable kit comprising one or more surgical instruments, are also disclosed.

### BACKGROUND

The invention described herein relates generally to medical instruments and, more particularly, to surgical instruments having a tool positioned at a working end thereof.

Arthroscopic surgery is a minimally-invasive surgical procedure during which an interior portion of the human body, such as a knee or shoulder joint, is examined or operated on using a arthroscope. An arthroscope is a type of endoscope or miniature camera that is passed through a cannula which is inserted into the joint through a small incision in the skin to enable a surgeon to view the joint and/or perform a surgical operation without fully opening the joint.

During arthroscopic surgery, a surgeon may also use an additional surgical instrument to push, pull or otherwise manipulate tissue in a patient's joint to evaluate tissue pathology. For example, the surgeon can make a diagnosis by observing how an additional instrument, such as a probe, pulls tissue apart or separates tissue from bone. The additional instrument may also be used as part of a surgical operation, such as shaving of bone tissue. The additional instrument is introduced into the patient through another incision in the skin and passed through a cannula that is adjacent to one used to introduce the arthroscope into the area of interest.

Sometimes it is difficult for the surgeon to see with the arthroscope underneath and around tissue, such as the undersurface of the patella or distal tibia, or the articular cartilage in the hip or shoulder. In the past, the surgeon might replace the arthroscope with another one having a different field of view (e.g., changing from an arthroscope with a 15° field of view to one with a 30° field of view), or the surgeon might even make an additional incision to insert the arthroscope into a different position to obtain an additional view. These approaches require additional time for the patient to be under anesthesia or in a tourniquet, sterilization time, and, in some cases, adds further trauma to and prolongs recovery time in the patient.

Accordingly, there is a need for a simple and sterilizable tool that would increase the field of view of an arthro scope without requiring a surgeon to make multiple incisions in a patent when performing a diagnosis and/or a surgical operation during arthroscopic surgery.

### SUMMARY

The invention described herein is a surgical instrument used in arthroscopic surgery with a reflecting feature, such as a mirror, adjacent to a tool disposed at the distal end of the instrument. The reflecting feature reflects the line of sight of an arthroscope during an arthroscopic surgery, improving the surgeon's view of a surgical site through the arthroscope. The surgical instrument of the current invention enables a surgeon to see aspects and regions previously difficult or impossible to see, as well as to aid in palpatory diagnosis of tissue pathology or in a surgical operation, without having to change portals within a joint of the human body, such as a hip, shoulder, or knee. For example, the invention described herein allows for full visualization of the undersurface of the patella during a knee operation, more visualization of the articular cartilage around the curve of the humeral head during a shoulder or hip operation, and/or complete visualization of the undersurface of the distal tibia during an ankle operation.

In one embodiment, the surgical instrument of the present invention comprises an elongated member having a proximal end, a distal end, and a longitudinal axis extending between the proximal and distal ends; a handle extending from the proximal end of the elongated member; a tool configured to manipulate tissue; and a reflecting member having a surface, the reflecting member disposed along the elongated member and adjacent to the tool, and the surface configured to reflect a line of sight of an arthroscope during arthroscopic surgery. The tool is either disposed at the distal end of the elongated member and has an axis transverse to the longitudinal axis of the elongated member or extends from the distal end of the elongated member and terminates at a distal terminus, the distal terminus being offset from the longitudinal axis of the elongated member.

As further described in the embodiments herein, the reflecting member of the surgical instrument is spaced at a distance from the tool based on the function of the tool and is disposed substantially perpendicular to the axis of the tool. The tool of the surgical instrument is either a probe, a curette or an elevator. The reflecting member is also formed integrally with the elongated member and has a substantially elongated shape. The long dimension of the reflecting member is parallel to the longitudinal axis of the elongated member, while the short dimension of the reflecting member is transverse to the longitudinal axis of the elongated member. The short dimension of the reflecting member is at least about 3 mm and more preferably about 5 mm to about 7.5 mm. The surface of the reflecting member comprises polished metal and is flat, planar, convex or concave. The reflected line of sight of the arthroscope by the reflecting member is substantially 180°.

The methods of the current invention include a method of increasing the line of sight of an arthroscope during arthroscopic surgery without removing or repositioning the arthroscope, the method comprising the steps of: 1) providing a surgical instrument as described above in an area adjacent to an arthroscope during arthroscopic surgery; and 2) reflecting a line of sight of the arthroscope with the surgical instrument.

An embodiment of the invention described herein also includes a combination of a kit and at least one surgical instrument, the combination comprising the kit in the form of a tray; each of the at least one surgical instrument being secured to the tray with a fastener, and each of the at least one surgical instrument being capable of independent removal from the tray; wherein the at least one surgical instrument comprises an elongated member having a proximal end, a distal end, and a longitudinal axis extending between the proximal and distal ends; a handle extending from the proximal end of the elongated member; a tool disposed at the distal end of the elongated member and configured to manipulate tissue; and a reflecting member having a surface, the reflecting member disposed along the elongated member, the reflecting member reflecting a line of sight of an arthroscope during arthroscopic surgery.

### BRIEF DESCRIPTION OF THE DRAWINGS

The foregoing and other objects, features and advantages will be apparent from the following more particular description of the examples, as illustrated in the accompanying drawings in which like reference characters refer to the same parts throughout the different views. The drawings are not necessarily to scale, emphasis instead being placed upon illustrating the principles of the examples.

FIG. 1 is a perspective illustration of an embodiment of the current invention;

FIGS. 2a-b are schematic views of various embodiments of the reflective member thereof;

FIG. 3 is a detailed photographic view of the reflective member;

FIG. 4 is a photographic view of various embodiments of the tool of the current invention;

FIG. 5 is a perspective view of an arthroscopic procedure of a knee joint using an arthroscope and an embodiment of current invention;

FIGS. 6a-b are photographic views of an embodiment of the current invention in use during arthroscopic surgery; and

FIG. 7 is a schematic representation of an embodiment of the kit of the current invention.

### DETAILED DESCRIPTION

In the description that follows, like components have been given the same reference numerals, regardless of whether they are shown in different examples. To illustrate an example(s) of the present invention in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and in the claims, the singular form of “a”, “an”, and “the” include plural referents unless the context clearly dictates otherwise.

As used in the specification and in the claims, for the purposes of describing and defining the invention, the terms “about” and “substantially” are used represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue.

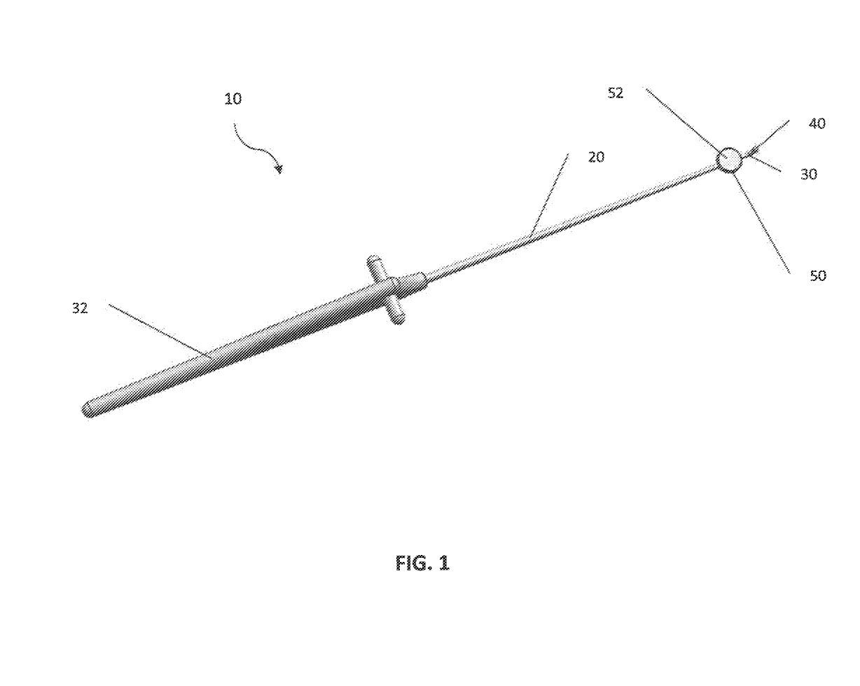

Referring now to FIG. 1, the surgical instrument 10 of this invention comprises an elongated member 20, a working end 30 located at the distal end of the elongated member 20, and a handle 32 located at the proximal end of the elongated member and opposite to the working end 30. A tool 40 is disposed at the working end 30 of the elongated member 20. The tool 40 may have an axis transverse to the longitudinal axis of the elongated member 20 but is not limited thereto.

The surgical instrument 10 in FIG. 1 also comprises a reflective member 50 disposed along the elongated member 20 and adjacent to the tool 40. The reflective member 50 can be formed integrally with the elongated member 20. The reflective member 50 has a surface 52 that has a degree of reflectivity operable to redirect or reflect an image of an area of interest of up to 180° to an arthroscope for viewing by a surgeon.

The placement of the reflective member 50 from the distance of the tool 40, as well as the degree of reflectivity of the surface 52, is variable and determined based on the desired function of the surgical instrument 10 or tool 40. The surface 52 of the reflecting member 50 may be comprised of polished metal or other suitable materials. The surface 52 of the reflecting member 50 may be disposed at any angle along the elongated member 20, while remaining substantially perpendicular to the axis of the tool 40.

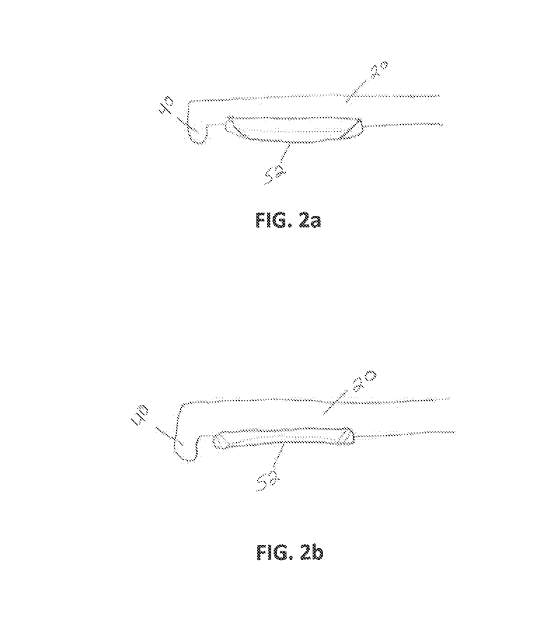

In FIG. 1, the surface 52 of the reflective member 50 is shown as flat or planar. However, the surface 52 may also be convex (FIG. 2a) or concave (FIG. 2b) to provide different degrees of reflectivity.

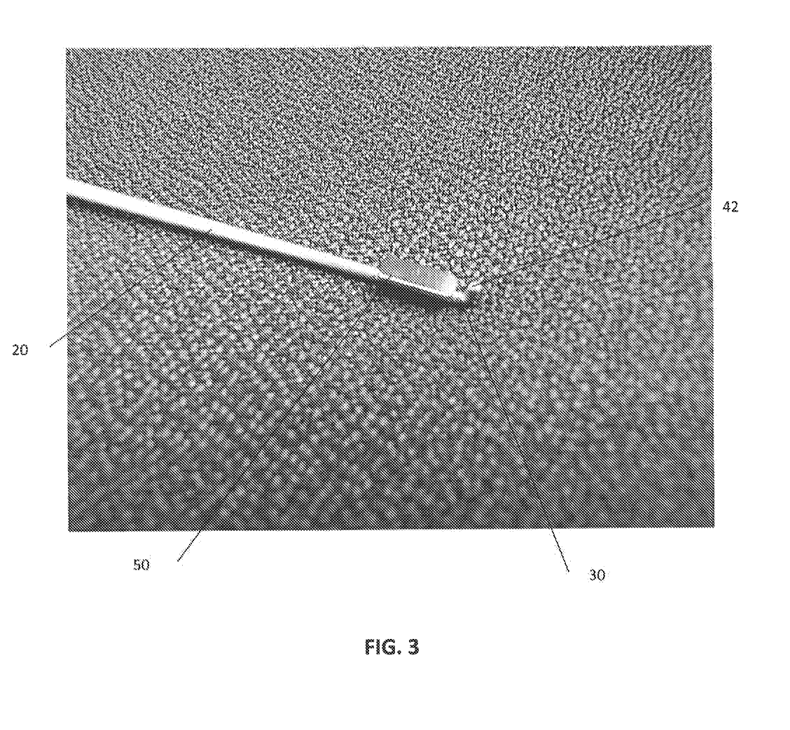

In FIG. 3, the working end 30 of the surgical instrument 10 is shown as a probe 42. The reflective member 50 can be any shape or configuration but is preferably rectangular, with the length of the reflecting member 50 being parallel to the longitudinal axis of the elongated member 20 and the width of the reflective member 50 being transverse to the longitudinal axis of the elongated member 20. The width of the reflective member 50 is variable but is preferably at least about 3 mm wide, and more preferably about 5 mm to about 7.5 mm, which corresponds to the widths of standard surgical cannulas used during arthroscopic surgery. The length of the reflective member 50 is also variable and is determined based on the desired function of the surgical instrument 10 or tool 40.

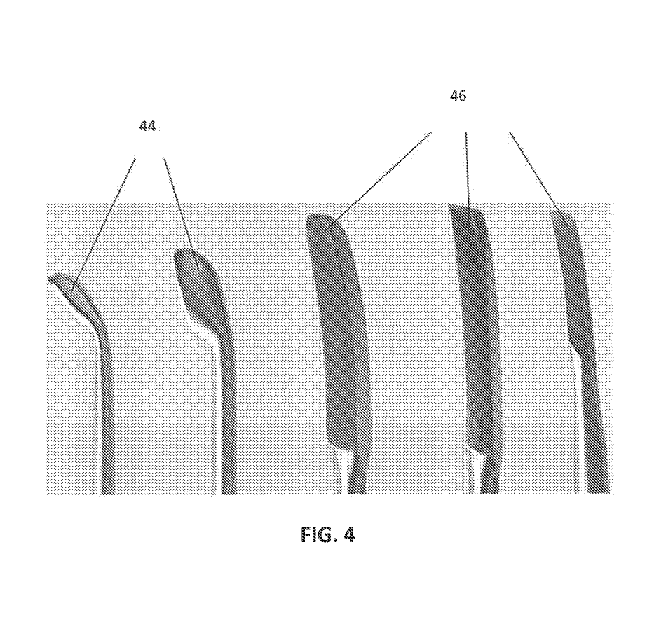

In both FIGS. 1 and 3, the tool 40 is shown for illustrative purposes as a probe 42; however, the tool 40 could be configured to provide any one or more of a plurality of surgical functions, such as ablating, burring, biting, sawing, shaving, suctioning and/or the like. FIG. 4 shows additional embodiments of the tool 40, which may be also be a curette 44 or an elevator 46.

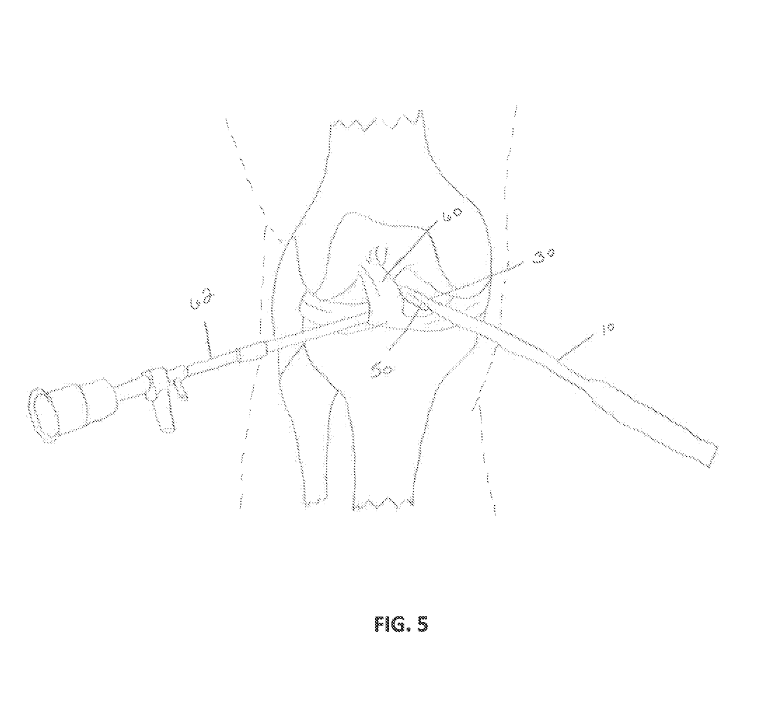

As shown in FIG. 5, when a surgeon performs an arthroscopic procedure, whether an examination or an operation, the surgeon selects the surgical instrument 10 that includes the teachings of the present invention, such as the probing device as shown in FIG. 1. After making incisions adjacent to an area of interest of the body, such as a knee joint 60, the surgeon independently inserts a portion of the arthroscope 62 and the working end 30 of the surgical instrument 10 into the incisions and through surgical cannulas (not shown) so that the portion of the arthroscope 62 and the working end 30 of the surgical instrument 10 substantially converge on the knee joint 60. If the surgeon is having difficulty viewing the area of interest directly via the arthroscope 62, the surgeon rotates the surgical instrument 10 so that the reflective member 50 reflects an image of the area of interest to the arthroscope 62.

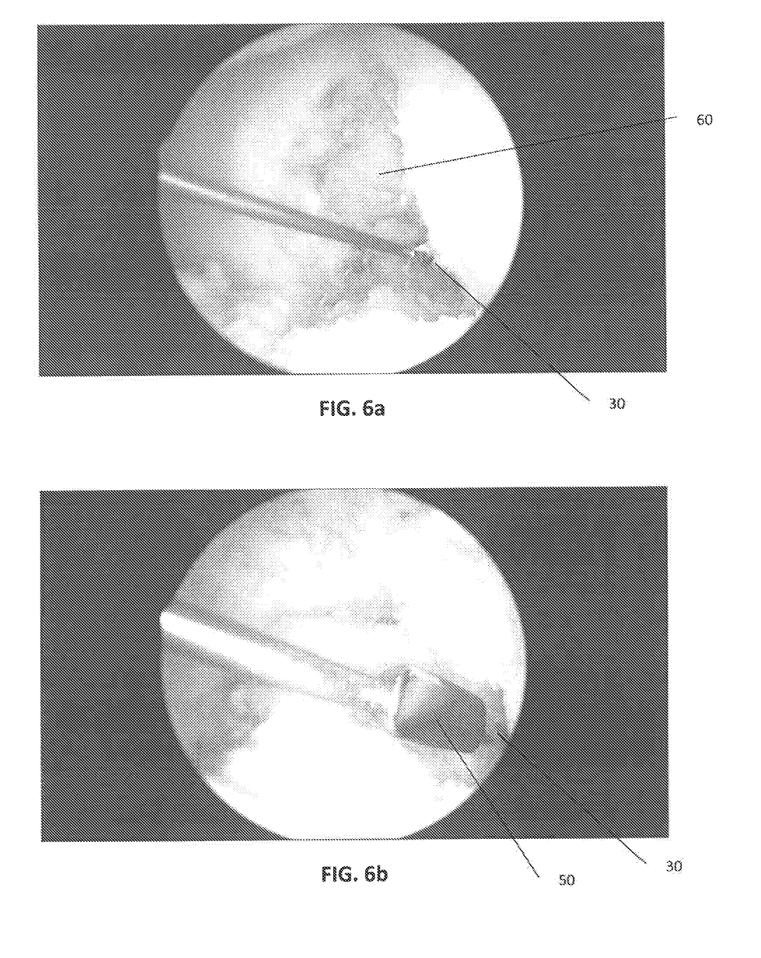

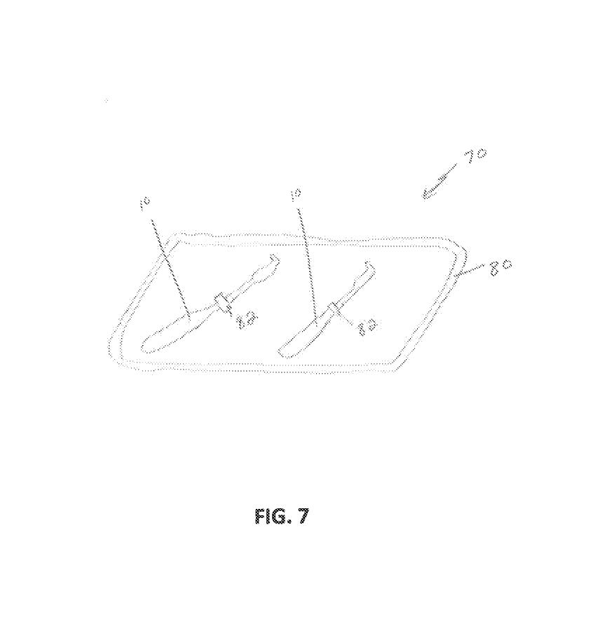

Two photographic illustrations of the surgical instrument 10 in use during arthroscopic surgery are shown in FIGS. 6a and 6b. FIG. 6a shows the working end 30 of the surgical instrument 10 advanced through the anteriormedial portal into the knee joint. The working end 30 enters anteriorly into the joint space between the two femoral condyles on either side of the knee joint 60. Using the arthroscope with a closer view of the working end 30, FIG. 6b shows the reflective member 50 in use by reflecting the image of the posterior aspect of the condyle, where the arthroscope would not normally be able to see. As such, the reflective member 50 allows for visualization around anatomical structures not normally accessible without changing the arthroscope portals or moving the arthroscope at inconvenient angles. FIG. 7 is a schematic illustration of a kit of the present invention. The kit 70 includes a tray 80 comprised of a material that is capable of undergoing a disinfection and/or sterilization procedure. One or more surgical instruments 10 (which may be probe, a curette or an elevator, or combinations thereof) can be included in the kit 70 and secured to the tray 80 by means of a suitable fastener 82.

These and other features and characteristics, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of claims.

1. A surgical instrument for arthroscopic surgery, the surgical instrument comprising:
an elongated member having a proximal end, a distal end, and a longitudinal axis extending between the proximal and distal ends;
a handle extending from the proximal end of the elongated member;
a tool disposed at the distal end of the elongated member, the tool having an axis transverse to the longitudinal axis of the elongated member, and configured to manipulate tissue; and
a reflecting member having a surface, the reflecting member disposed along the elongated member and adjacent to the tool, the surface configured to reflect a line of sight of an arthroscope during arthroscopic surgery.

2. The surgical instrument of claim 1, wherein reflecting member is spaced at a distance from the tool based on the function of the tool.

3. The surgical instrument of claim 1, wherein the surface of the reflecting member is disposed substantially perpendicular to the axis of the tool.

4. The surgical instrument of claim 1, wherein the tool is a probe.

5. The surgical instrument of claim 1, wherein the tool is a curette.

6. The surgical instrument of claim 1, wherein the reflecting member is formed integrally with the elongated member.

7. The surgical instrument of claim 1, wherein the reflecting member has a substantially elongated shape.

8. The surgical instrument of claim 1, wherein a long dimension of the reflecting member is parallel to the longitudinal axis of the elongated member.

9. The surgical instrument of claim 1, wherein a short dimension of the reflecting member is transverse to the longitudinal axis of the elongated member.

10. The surgical instrument of claim 9, wherein the short dimension of the reflecting member is at least about 3 mm.

11. The surgical instrument of claim 10, wherein the short dimension of the reflecting member is about 5 mm to about 7.5 mm.

12. The surgical instrument of claim 1, wherein the surface of the reflecting member comprises polished metal.

13. The surgical instrument of claim 1, wherein the surface of the reflecting member is flat or planar.

14. The surgical instrument of claim 1, wherein the surface of the reflecting member is convex.

15. The surgical instrument of claim 1, wherein the surface of the reflecting member is concave.

16. The surgical instrument of claim 1, wherein the reflected line of sight of the arthroscope is substantially 180°.

17. A method of increasing the line of sight of an arthroscope during arthroscopic surgery without removing or repositioning the arthroscope, the method comprising the steps of:
providing a surgical instrument according to claim 1 in an area adjacent to the arthroscope during arthroscopic surgery; and
reflecting a line of sight of the arthroscope with the surgical instrument.

18. The combination of a kit and at least one surgical instrument, the combination comprising:
the kit in the form of a tray; each of the at least one surgical instrument being secured to the tray with a fastener, and each of the at least one surgical instrument being capable of independent removal from the tray;
wherein the at least one surgical instrument comprises:
an elongated member having a proximal end, a distal end, and a longitudinal axis extending between the proximal and distal ends;
a handle extending from the proximal end of the elongated member;
a tool disposed at the distal end of the elongated member, the tool having an axis transverse to the longitudinal axis of the elongated member, and configured to manipulate tissue; and
a reflecting member having a surface, the reflecting member disposed along the elongated member, the reflecting member reflecting a line of sight of an arthroscope during arthroscopic surgery.

19. The combination of claim 18, wherein the at least one surgical instrument is a probe

20. The combination of claim 18, wherein the at least one surgical instrument is a curette.

21. The combination of claim 18, wherein the tray and the at least one surgical instrument are adapted to undergo a disinfection or sterilization procedure.

22. A surgical instrument for arthroscopic surgery, the surgical instrument comprising:
an elongated member having a proximal end, a distal end, and a longitudinal axis extending between the proximal end and distal end;
a handle extending from the proximal end of the elongated member;
a tool extending from the distal end of the elongated member and terminating at a distal terminus, the distal terminus is offset from the longitudinal axis of the elongated member; the tool configured to manipulate tissue; and
a reflecting member disposed along the elongated member and adjacent to the tool, the reflecting member having a surface, the surface configured to reflect a line of sight of an arthroscope during arthroscopic surgery.

23. The surgical instrument of claim 22, wherein the tool is an elevator.

24. A method of increasing the line of sight of an arthroscope during arthroscopic surgery without removing or repositioning the arthroscope, the method comprising the steps of:
providing a surgical instrument according to claim 22 in an area adjacent to the arthroscope during arthroscopic surgery; and
reflecting a line of sight of the arthroscope with the surgical instrument.

25. The method of claim 24, wherein the tool is an elevator.

26. The combination of a kit and at least one surgical instrument, the combination comprising:
the kit in the form of a tray; each of the at least one surgical instrument being secured to the tray with a fastener, and each of the at least one surgical instrument being capable of independent removal from the tray;
wherein the at least one surgical instrument comprises:
an elongated member having a proximal end, a distal end, and a longitudinal axis extending between the proximal and distal ends;
a handle extending from the proximal end of the elongated member;
a tool disposed at the distal end of the elongated member, the tool having an axis transverse to the longitudinal axis of the elongated member, and configured to manipulate tissue; and
a reflecting member having a surface, the reflecting member disposed along the elongated member, the reflecting member reflecting a line of sight of an arthroscope during arthroscopic surgery.

27. The combination of claim 26, wherein the at least one tool is an elevator.
